(19) United States
(12) Patent Application Publication
(10) Pub. No.: US 2015/0174122 A1
(43) Pub. Date: Jun. 25, 2015

(54) METHODS FOR TREATING EYE DISORDERS USING OPIOID RECEPTOR ANTAGONISTS

(71) Applicant: Remedeye Inc., Wilmington, DE (US)

(72) Inventor: Moshe ROGOSNITZKY, Kiryat Ye'arim (IL)

(21) Appl. No.: 14/636,211

(22) Filed: Mar. 3, 2015

Related U.S. Application Data
(62) Division of application No. 13/792,735, filed on Mar. 11, 2013.

(30) Foreign Application Priority Data
Jan. 27, 2013 (IL) 224422

(51) Int. Cl. A61K 31/485; A61K 9/00

(52) U.S. Cl. CPC A61K 31/485; A61K 9/0048

(57) ABSTRACT

A method comprising: topically administering to eye/s of a subject in need of treatment for a disorder selected from the group consisting of pterygium, pinguecula and pink eye a composition comprising an effective amount of an active ingredient selected from the group consisting of naltrexone, naloxone or nalmefene or a pharmaceutically acceptable salt thereof and a pharmaceutically acceptable carrier.

### CROSS REFERENCE TO RELATED APPLICATIONS

This patent application is a divisional application of U.S. Ser. No. 13/792,735 filed Mar. 11, 2013, which is currently pending and claimed priority under 35 U.S.C. §365(c) to Israel Patent Application No. 224422 filed Jan. 27, 2013; each of which is hereby incorporated by reference in its entirety.

### FIELD AND BACKGROUND OF THE INVENTION

The present invention relates to methods for treating eye disorders using opioid receptor antagonists.

As known in the art, the medical condition referred to as “dry eye” is a disorder of the tear film due to tear deficiency or excessive tear evaporation which causes damage to the interpalpebral ocular surface associated with symptoms of ocular discomfort. Chronic dryness leads to pain and irritation that is often debilitating to the subject, preventing the performance of normal daily activities such as reading and driving. Dry eye is increasing in prevalence as the population ages. Approximately 4.9 million Americans 50 years old and older have dry eye. Many more have less severe symptoms notable only during contact with adverse contributing factors such as low humidity or contact lens wear, as well as the 21 million individuals with diabetes. The number of women affected with dry eye appears to exceed that of men.

Currently, dry eye includes two major classes: (i) aqueous tear-deficient dry eye (ADDE), and (ii) evaporative dry eye (EDE). ADDE refers mainly to a failure of sufficient tear secretion due to lacrimal dysfunction. ADDE has two major subclasses, Sjogren's Syndrome dry eye (SSDE) and non-SS dry eye (such as in Graft-versus-Host Disease (GvHD) or in diabetes mellitus). EDE may be: (i) intrinsic, due to diseases affecting lid structures or dynamics, or (ii) extrinsic, in which ocular surface disease occurs due to some extrinsic exposure, such as topical drug preservatives, contact lens wear, pterygium, or vitamin A deficiency. The boundary between these two categories is inevitably blurred.

Current therapies for dry eye include tear supplementation (e.g., lubricants), tear retention, tear stimulation, tear substitutes, anti-inflammatory therapy, and essential fatty acids. Over-the-counter lubricants are most frequently prescribed by vision specialists; however, lubricants offer only temporary relief, can be expensive, and need to be taken for life. The cause of dry eye is not treated with lubricants.

The human cornea consists of epithelial cells, Bowman's membrane, corneal stroma, Descemet's membrane, and endothelial cells. The cornea is normally avascular tissue, and receives nutrients from the aqueous humor in the anterior chamber of eye instead of blood vessels. The cornea also lacks immunocytes. Therefore, the cornea is a very special tissue as compared to other tissues.

The term “corneal ulcer” usually refers to the medical condition in which the corneal stroma, or simply stroma (which mainly consists of collagen) is lysed and deleted by the activation and hypersecretion of collagenolytic enzyme. The collagenolytic enzyme causing corneal ulcer, bacterial collagenase, and matrix metalloproteases (MMPs) are known to be involved in the ulcerative process.

When the cornea is infected with bacteria, bacterial collagenase is secreted, which directly degrades collagen in corneal stroma, and causes corneal ulcer. Simultaneously, microorganisms such as bacteria secrete other enzymes and toxins, and these factors become biological signals causing activation of corneal stromal cells (sometimes referred to as corneal fibroblasts). Other causes include viral, fungal, parasitic infection, or traumatic injury.

The changes in the extracellular environment caused by the degradation of stromal collagen promote ulcers. Such conditions produce a vicious circle of activation of corneal stromal cells and degradation of corneal stroma. When the bacteria are killed by antibiotics, secretion of bacterial collagenase is suppressed, and direct corneal stroma degradation due to the bacteria is suppressed.

However, since most antibiotics cannot suppress activation of corneal stromal cell caused by the biological signals once transmitted from bacteria to corneal stromal cells, progression of ulcer is clinically observed from time to time. The factor to suppress such a vicious circle is considered to be anti-inflammatory agents such as steroids, as reported in (Teruo et al., ed., “Monthly ophthalmology therapeutic practice (vol. 79) pharmacotherapeutics of corneal and conjunctival diseases,” 1st printing, Bunkodo, January 2002, p. 9-11).

In the corneal epithelium, cells are columnar at the basal section but become flatter toward the surface. The epithelial cells are divided at the basal section, and gradually migrate upwards to finally be shed off and carried away by tears. The corneal endothelial cells do not regenerate because they do not undergo cell division. The delayed treatment or chronic state of corneal and conjunctival diseases such as dry eye, corneal ulcer, corneal erosion, pterygium, and keratitis damages the structures and functions of not only the epithelium, but also stroma and endothelium, and seriously impairs vision and barrier function.

The corneal/conjunctival diseases, including a repeated erosion of the cornea and a prolonged corneal epithelial deficiency, are associated with such disorders. The repairing process of the corneal/conjunctival epithelial disorders involves the coverage of the epithelial deficiency by the migration of corneal epithelial cells, followed by a subsequent cell division and differentiation, resulting in reconstitution of normal cornea and conjunctiva.

For example, dry eyes also seriously impair vision and the barrier functions. This can occur when dry eyes result from, for example, (i) Sjogren's syndrome, (ii) Stevens Johnson syndrome, (iii) meibomian gland function insufficiency, (iv) VDT (visual display terminal) syndrome, (v) ophthalmological operations (e.g., cataract surgery, keratoplasty, refractive surgery); (vi) GvHD; (vii) keratoconjunctivitis sicca; and (viii) corneal disorders such as superficial punctate keratopathy (SPK), a fine, spot-like multiple epithelial deficiency occurring on the cornea epithelium (observed in, for example, drug-induced corneal epithelial disorders, neuroparalytic keratitis, diabetic keratopathy, and allergic conjunctivitis). Further, outbreaks of corneal epithelial damage such as superficial punctate keratopathy (SPK) among contact lens wearers have become a big problem in recent years.

It would be desirable to have methods for treating eye disorders using opioid receptor antagonists. Such methods and treatment indications would, inter alia, overcome the problems mentioned above associated with such ailments.

### SUMMARY OF THE INVENTION

It is the purpose of the present invention to provide methods for treating eye disorders using opioid receptor antagonists.

In the interest of clarity, the term “eye disorder” is specifically defined for use herein to include, but not be limited to, any ailment of Scleritis, Graft-versus-Host Disease (GvHD), keratitis, corneal ulcer, corneal abrasion, snow blindness, Thygeson's superficial punctuate keratopathy, corneal neovascularization, Fuch's dystrophy, keratoconus, keratoconjunctivitis sicca (dry eye), iritis, corneal anesthesia, red eye, pink eye, keratomycosis, xeropthalmia, conjunctivitis, diabetic retinopathy, optic neuritis, orbital cellulitis, retinoblastoma, uveitis, pterygium, keratopathy, and Pingueculae.

Furthermore, it is noted that the term “exemplary” is used herein to refer to examples of embodiments and/or implementations, and is not meant to necessarily convey a more-desirable use-case. Similarly, the term “preferred” is used herein to refer to an example out of an assortment of contemplated embodiments and/or implementations, and is not meant to necessarily convey a more-desirable use-case. Therefore, it is understood from the above that “exemplary” and “preferred” may be applied herein to multiple embodiments and/or implementations.

Opioids are drugs or endogenous substances that have actions similar to morphine. Endogenous or exogenous opioids exert their biological function in the body through binding to opioid receptors. An opioid receptor antagonist is a chemical that competitively binds to opioid receptors, thus displacing either endogenous or exogenous opioids.

Commonly-used opioid antagonist drugs such as naloxone, naltrexone, and nalmefene are competitive antagonists that bind to the opioid receptors with higher affinity than agonists, but do not activate the receptors. This effectively blocks the receptor, preventing the body from responding to opioids and endorphins.

Naloxone, naltrexone, and nalmefene are pure opioid receptor antagonists, meaning that they exert an opioid-blocking effect in all opioid receptors. Mixed or partial antagonists act as antagonists in certain receptors, and as agonists in others, thus giving a simultaneous, combined opioid and anti-opioid effect.

Embodiments of the present invention provide methods for treating eye disorders using opioid receptor antagonists, particularly, naltrexone, naloxone, and nalmefene. Naltrexone is a highly-specific opioid antagonist which has a high affinity for opioid receptor sites. Naltrexone competitively displaces opioid agonists, such as opium, methadone, heroin, morphine, endogenous opioid peptides, beta-endorphin, and met-enkephalin, if one of these compounds is present. The compound has few intrinsic actions besides its opioid-blocking properties. Naltrexone does produce some pupillary constriction by an unknown mechanism, but does not cause any physiological tolerance or dependence. It is not known to block the effects of other classes of drugs besides opioids. However, naltrexone appears to block some of the euphoriant actions of alcohol, presumably due to its blockade of opioid receptors.

Naltrexone (NTX) is rapidly absorbed, with peak blood levels achieved about one hour after oral administration. NTX has a relatively short plasma half-life of four hours. It is primarily metabolized in the liver to a metabolite, 6-β-naltrexol, which has a plasma half-life of about ten hours, and is also an opioid antagonist. Approximately 20% of the active metabolite is bound to plasma protein, and is distributed widely, with relatively high amounts in the brain, fat, spleen, heart, testes, kidney and urine. Naltrexone and 6-β-naltrexol undergo enterohepatic recycling, and are excreted primarily by the kidney. Less than 1% of naltrexone is excreted unchanged.

Naloxone is similar to naltrexone, having a similar function on opioid receptors; however, naloxone is very short-acting, as opposed to the long-acting effect of NTX. Naloxone eye drops were reported to reverse the miosis in runners, having implications for an endogenous opiate test. Test results suggested that exercise generated endogenous opiates which cause pupillary miosis, and that applying topical naloxone to one eye can block this exercise pupillary effect resulting in ipsilateral mydriasis.

However, other studies on the use of topical ophthalmic naloxone to detect physiological opioid dependence have shown that a naloxone eye-drop test may not be a useful tool in determining the presence of neuroadaptation to opioids.

Nalmefene is an opiate derivative similar both in structure and activity to the opioid antagonist naltrexone. Advantages of nalmefene relative to NTX include longer half-life, greater oral bioavailability, and no observed dose-dependent liver toxicity.

In the prior art, Zagon et al. published a research report (Brain Research, 798 (1998) 254-260) entitled, “Re-epithelialization of the rat cornea is accelerated by blockade of opioid receptors.” On page 258, right column, the authors conclude, “Although systemically administered NTX markedly influenced the incidence of re-epithelialization, differences were not detected between rats receiving topical application of NTX or vehicle.” The vehicle used was sterile water. Thus, it was shown that topical application of NTX in sterile water was not effective.

On page 241, left column, of a review study (Brain Research Bulletin, 81 (2010) 236-247) entitled, “Diabetic keratopathy and treatment by modulation of the opioid growth factor (OGF)-OGF receptor (OGFr) axis with naltrexone: A review,” McLaughlin et al. report, “Studies to determine the toxicity of NTX applied topically were conducted on Normal and type 1 diabetic rats using intact corneas [115], as well as rats that had abraded corneas [62]. Using intact corneas, concentrations of 10⁻³ to 10⁻⁷M NTX as well as vehicle (Vigamox, Alcon) were applied 4 times daily for 7 days.” Similar findings involving NTX/Vigamox combination treatment indications are recited in U.S. Pat. No. 8,314,118 by Zagon, McLaughlin, and Sassani.

Vigamox is the brand name for moxifloxacin (MXF), a fourth-generation synthetic fluoroquinolone antibacterial agent (methoxyfluoroquinolone). MXF also goes by the brand name Avelox. In a study conducted by Choi et al. (Antimicrob Agents Chemother., 2003 December; 47(12): 3704-3707), MXF was shown to inhibit the production of inflammatory proteins such as tumor necrosis factor alpha (TNF-α) and/or interleukin-6 (IL-6), and to reduce the population of cells positive for CD-14 and TNF-α and for CD-14 and IL-6 among the LPS- or LTA-stimulated, human peripheral blood mononuclear cells (PBMCs).

Weiss et al. (Antimicrob Agents Chemother., 2004 Jun; 48(6):1974-82) further reported MXF's anti-inflammatory properties in monocytes and THP-1 cells via the inhibition of NF-κB, ERK, and JNK activation. The direct anti-inflammatory effects of moxifloxacin on a lung epithelial cell line were disclosed by Li et al. (Respirology, 2012 Aug; 17(6):997-1005) finding that MXF inhibits intracellular signaling, iNOS expression, and NO secretion. It was postulated that dexamethasone may also have additional or synergistic effects with MXF. In WIPO Patent Publication No. WO/2010042549 entitled, “Inhalation of levofloxacin for reducing lung inflammation,” Dudley et al. disclose that MXF can inhibit secretion of pro-inflammatory factor such as IL-8, IL-6, ERK1/2, JNK, and NF-κB in human lung epithelial cells.

Chen et al. (Br J Pharmacol., 2012 Oct. 16) found that MXF modifies corneal fibroblast-to-myofibroblast differentiation. MXF retarded HCF-containing gel contractility and α-SMA filament formation following TGF-β1 stimulation, and blocked expression of Smad2, phospho-Smad2-Ser467, and TGFBR1 under TGF-β1 incubation, as well as enhanced Smad7 expression in TGF-β1-incubated HCFs, but did not interfere with TGF-β1-triggered Smad2 nuclear translocation or Smad4 expression.

Therefore, it is clear from the above references that MXF is an active chemical agent with properties and interactions that go well beyond typical antimicrobial effects.

Additionally, in a study by Etminan et al. (JAMA, 2012 Apr. 4; 307(13):1414-9), the authors summarize their findings as being “consistent with an association between fluoroquinolone [of which MXF is a member] use and the risk of retinal detachment.” Ocular (aqueous humoral) levels of MXF achieved through oral administration and associated with retinal detachment were comparable to the ocular (aqueous humoral) levels achieved from topical ocular MXF as used in the study by McLaughlin et al. (cited above) using an NTX/Vigamox formulation.

Thus, it can be concluded from the above (and studies of others) that use of MXF (or its class of antibiotics) causes a four-fold increase in the risk of retinal detachments when taken orally, and that topical MXF use (as in the NTX/Vigamox study) poses at least a comparable risk. Furthermore, prolonged use of MXF for eye disorders poses an additional risk of contracting fungal infections. Moreover, indiscriminate use of antibiotics in general is the main known cause of antibiotic resistance.

All of the above contraindications would make such medical treatment for eye disorders undesirable, even though MXF provides anti-inflammatory activity, whether in isolation or synergistically with NTX. Furthermore, the lack of activity for NTX in sterile water reported by Zagon et al. (cited above) raises the uncertainty regarding the broad applicability of NTX for such treatment indications.

From a practical commercial perspective, 31 CFR §300.50 of the United States Food and Drug Administration's policy, regarding fixed combination dosage form prescription drugs for humans, states that two or more drugs may be combined in a single dosage form when (i) each component makes a contribution to the claimed effects, and (ii) the dosage of each component (amount, frequency, duration) is such that the combination is safe and effective for a significant patient population requiring such concurrent therapy as defined in the labeling for the drug.

The European Medicines Agency has similar guidelines to the FDA, stating that the development of fixed-combination medicinal products will reflect the intended use and the intended indication. The proposed combination should always be based on valid therapeutic principles. In addition, it is necessary to assess the potential advantages (e.g., product rapidly effective, higher efficacy or equal efficacy, and better safety) in the clinical situation against possible disadvantages (e.g., cumulative toxicity) for each fixed combination product and for each dose of the fixed combination product.

Thus, due to the current state-of-the-art understanding as described above regarding the uncertainty of NTX and MXF activity and combination interaction, regulatory hurdles pose an additional obstacle for NTX/MXF combination treatment indications.

In accordance with aspects of the present invention, opioid receptor antagonists were found to be effective in treating ocular medical conditions when applied topically in physiological saline formulations. In particular, topical application of opioid receptor antagonists may serve to treat dry eye caused by, for example, Graft-versus-Host Disease (GvHD), diabetes, allergic conjunctivitis, contact lens-related dry eye, and Sjorgen's syndrome.

In an exemplary embodiment of the present invention, topical opioid receptor antagonists may also be used to treat corneal ulcers resulting from, for example: viral infection, bacterial infection, fungal infection, injury resulting from wearing contact lenses, traumatic injury, and parasite infection. Moreover, topical opioid receptor antagonists may also be used for the treatment of pterygium, corneal anesthesia, and corneal neovascularization.

Therefore, according to the present invention, there is provided for the first time a method for treating eye disorders using opioid receptor antagonists, the method including the step of: (a) administering an effective amount of a topically-administered opioid receptor antagonist in the absence of moxifloxacin.

Preferably, the topically-administered opioid receptor antagonist is formulated as a solution.

Preferably, the topically-administered opioid receptor antagonist is at least one agent selected from the group consisting of: naltrexone, naloxone, nalmefene, and a pharmaceutically-acceptable salt thereof.

Preferably, the effective amount corresponds to a concentration of at least about 10⁻⁷ molarity.

Preferably, the effective amount is based on a treatment administration of at least once every other day.

These and further embodiments will be apparent from the detailed description and examples that follow.

### DESCRIPTION OF THE PREFERRED EMBODIMENTS

The present invention relates to methods for treating eye disorders using opioid receptor antagonists. The aspects, uses, and advantages for such methods and treatment indications, according to the present invention, may be better understood with reference to the accompanying description.

The description is not to be taken in a limiting sense, but is made merely for the purpose of illustrating the general principles of the invention, since the scope of the present invention is best defined by the appended claims. Exemplary embodiments of the present invention are detailed below in the following exemplary formulations.

### Exemplary Formulaton A

Naltrexone eye drops were prepared as follows. 1 mg naltrexone hydrochloride USP was weighed and diluted in 265 ml saline (0.9% w/v sodium chloride in sterile water). This gave a solution containing 3.77 mcg of naltrexone per ml. Using a dropper, one drop (equivalent to approximately 0.05 ml) was applied to the eye.

### Exemplary Formulaton B

Naltrexone eye drops were prepared as follows. 1 mg naltrexone hydrochloride USP was weighed and diluted in 132.5 ml saline (0.9% w/v sodium chloride in sterile water). This gave a solution containing 7.54 mcg of naltrexone per ml. Using a dropper, one drop (equivalent to approximately 0.05 ml) was applied to the eye.

### Exemplary Formulaton C

Naloxone eye drops were prepared as follows. 0.5 mg naloxone hydrochloride USP was weighed and diluted in 265 ml saline (0.9% w/v sodium chloride in sterile water). This gave a solution containing 1.886 mcg of naloxone per ml. Using a dropper, one drop (equivalent to approximately 0.05 ml) was applied to the eye.

### Exemplary Formulaton D

Naloxone eye drops were prepared as follows. 0.5 mg naloxone hydrochloride USP was weighed and diluted in 132.5 ml saline (0.9% w/v sodium chloride in sterile water). This gave a solution containing 3.77 mcg of naloxone per ml. Using a dropper, one drop (equivalent to approximately 0.05 ml) was applied to the eye.

### Exemplary Formulaton E

Nalmefene eye drops were prepared as follows. 1 mg nalmefene hydrochloride USP was weighed and diluted in 265 ml saline (0.9% w/v sodium chloride in sterile water). This gave a solution containing 3.77 mcg of nalmefene per ml. Using a dropper, one drop (equivalent to approximately 0.05 ml) was applied to the eye.

### Exemplary Formulaton F

Nalmefene eye drops were prepared as follows. 1 mg nalmefene hydrochloride USP was weighed and diluted in 132.5 ml saline (0.9% w/v sodium chloride in sterile water). This gave a solution containing 7.54 mcg of nalmefene per ml. Using a dropper, one drop (equivalent to approximately 0.05 ml) was applied to the eye.

### RESULTS

Two human males and two human females suffering from GvHD-related dry eye were treated with one drop of Formulation A (naltrexone) bilaterally twice daily. Relief from the dry-eye symptoms was attained within an hour. The patients required subsequent application twice daily. After 10 days of use, redness in the eye (or pink eye) disappeared.

A human male suffering from diabetes-related dry eye was treated with one drop of Formulation B (naltrexone) bilaterally twice daily. Relief from the dry-eye symptoms was attained within half an hour. The patient required subsequent application twice daily. After 10 days of use, redness in the eye (or pink eye) disappeared.

A human female suffering from diabetes-related dry eye was treated with one drop of Formulation B (naltrexone) bilaterally once every other day. Relief from the dry-eye symptoms was attained within half an hour. The patient required subsequent application once every other day. After 10 days of use, redness in the eye (or pink eye) disappeared.

A human male suffering from a viral eye infection with corneal abrasion (i.e., the onset of a corneal ulcer) was treated with one drop of Formulation A (naltrexone) bilaterally twice daily. Exudation ceased within 6 hours. The patient required subsequent application twice daily. After 2 days of use, redness in the eye (or pink eye) disappeared, and the eye was complete healed within 5 days.

A human female suffering in one eye from pterygium, with related dry eye and pink eye, was treated with one drop of Formulation B (naltrexone) twice daily. Relief from the dry-eye symptoms was attained within half an hour. The patient required subsequent application twice daily. After 14 days of use, redness in the eye (or pink eye) disappeared. After 2 months of use, the ptyregium shrank by about 50%, and continued to decrease in size with ongoing use.

A human male suffering from a corneal ulcer in one eye was treated with one drop of Formulation A (naltrexone) twice daily. Relief from pain and irritation was attained within 36 hours. The patient required subsequent application twice daily. After 7 days of use, the ulcer had healed completely.

Two males suffering from diabetes-related corneal anesthesia were treated with one drop of Formulation B (naltrexone) daily. Symptoms of corneal anesthesia began improving within 2 days. The patients required subsequent application daily. After 2 weeks of use, the patients reported complete relief of symptoms.

One male suffering from diabetes-related neovascularization was treated with one drop of Formulation B (naltrexone) twice daily. The patient required subsequent application twice daily. When examined after 3 weeks of use, the abnormal vessels were no longer visible by slit-lamp photography examination.

Two human females suffering from a viral eye infection with corneal abrasion (i.e., the onset of a corneal ulcer) were treated with one drop of Formulation C (naloxone) bilaterally twice daily. Exudation ceased within 3-6 hours. The patients required subsequent application twice daily. After 2 days of use, redness in the eye (or pink eye) disappeared, and the eyes were completely healed within 5-6 days.

Two human females suffering from a corneal ulcer in one eye were treated with one drop of Formulation C (naloxone) twice daily. Relief from pain and irritation was attained within 24 hours. The patients required subsequent application twice daily. After an average of 7 days of use, the ulcers had healed completely.

Five human males suffering from diabetes-related dry eye were treated with one drop of Formulation D (naloxone) bilaterally twice daily. Relief from the dry-eye symptoms was attained on average within half an hour. The patients required subsequent application twice daily. After an average of 8 days of use, redness in the eye (or pink eye) completely disappeared.

Four females suffering from diabetes-related corneal anesthesia were treated with one drop of Formulation D (naloxone) daily. The patients required subsequent application daily. Symptoms of corneal anesthesia started improving within 2 days. After approximately 8 days of use, the patients reported complete relief of symptoms.

Three males suffering from diabetes-related neovascularization were treated with one drop of Formulation D (naloxone) twice daily. The patients required subsequent application twice daily. When examined after an average of 16 days of use, the abnormal vessels were no longer visible by slit-lamp photography examination.

Five human females suffering from GvHD-related dry eye were treated with one drop of Formulation E (nalmefene) bilaterally twice daily. Relief from the dry-eye symptoms were attained within one to two hours. The patients required subsequent application twice daily. After an average of 6 days, redness in the eye (or pink eye) disappeared.

Three human females suffering from GvHD-related dry eye were treated with one drop of Formulation E (nalmefene) bilaterally once every other day. Relief from the dry-eye symptoms was attained within one to two hours. The patients required subsequent application once every other day. After an average of 6 days, redness in the eye (or pink eye) disappeared.

Two human males suffering from a viral eye infection with corneal abrasion (i.e., the onset of a corneal ulcer) were treated with one drop of Formulation E (nalmefene) bilaterally twice daily. Exudation ceased within 4-6 hours. The patients required subsequent application twice daily. After 2 days of use, redness in the eye (or pink eye) disappeared, and the eyes were completely healed within 4-5 days.

A human female suffering from a corneal ulcer in one eye was treated with one drop of Formulation E (nalmefene) twice daily. Relief from pain and irritation was attained within 24 hours. The patient required subsequent application twice daily. After 7 days of use, the ulcer had healed completely.

Three human males suffering from diabetes-related dry eye were treated with one drop of Formulation F (nalmefene) bilaterally twice daily. Relief from the dry-eye symptoms was attained on average within half an hour. The patients required subsequent application twice daily. After an average of 10 days of use, redness in the eye (or pink eye) completely disappeared.

Two human males suffering from diabetes-related dry eye were treated with one drop of Formulation F (nalmefene) bilaterally once every other day. Relief from the dry-eye symptoms was attained on average within an hour. The patients required subsequent application once every other day. After an average of 9 days of use, redness in the eye (or pink eye) completely disappeared.

A human male suffering in one eye from pterygium, with related dry eye and pink eye, was treated with one drop of Formulation F (nalmefene) twice daily. Relief from the dry-eye symptoms was attained within an hour. The patient required subsequent application twice daily. After 14 days of use, redness in the eye (or pink eye) disappeared. After 9 weeks of use, the ptyregium shrank by about 60%, and continued to decrease in size with ongoing use.

Three males suffering from diabetes-related corneal anesthesia were treated with one drop of Formulation F (nalmefene) daily. The patients required subsequent application daily. Symptoms of corneal anesthesia began improving within 2 days. After approximately 10 days of use, the patients reported complete relief of symptoms.

Two males suffering from diabetes-related neovascularization were treated with one drop of Formulation F (nalmefene) twice daily. The patients required subsequent application twice daily. After 18 days of use, the abnormal vessels were no longer visible by slit-lamp photography examination.

While the present invention has been described with respect to a limited number of embodiments, it will be appreciated that many variations, modifications, and other applications of the present invention may be made.

1. A method comprising:
topically administering to eye/s of a subject in need of treatment for a disorder selected from the group consisting of pterygium, pinguecula and pink eye a composition comprising an effective amount of an active ingredient selected from the group consisting of naltrexone, naloxone or nalmefene or a pharmaceutically acceptable salt thereof and a pharmaceutically acceptable carrier.

2. A method according to claim 1, wherein the effective amount corresponds to 50-100 microliters at a concentration of at least about 10⁻⁷ Molar.

3. A method according to claim 1, wherein the effective amount corresponds to 50-100 microliters at a concentration of about 10⁻³ Molar or less.

4. A method according to claim 1, wherein the composition does not contain moxifloxacin or a salt thereof.

5. A method according to claim 1, wherein said administration is at a frequency of at least once per day.

6. A method according to claim 1, comprising identifying said subject.

7. A method according to claim 1, wherein said administration is at a frequency of less than once per day.

8. A method comprising:
topically administering to eye/s of a subject in need of treatment for corneal ulcer involving the stroma a composition comprising an effective amount of an active ingredient selected from the group consisting of naltrexone, naloxone or nalmefene or a pharmaceutically acceptable salt thereof and a pharmaceutically acceptable carrier.

9. A method according to claim 8, wherein the composition does not contain moxifloxacin or a salt thereof. wherein the effective amount corresponds to 50-100 microliters at a concentration of at least about 10⁻⁷ Molar.

10. A method according to claim 8, wherein the effective amount corresponds to 50-100 microliters at a concentration of about 10⁻³ Molar or less.

11. A method according to claim 8, wherein the composition does not contain moxifloxacin or a salt thereof.

12. A method according to claim 8, wherein said administration is at a frequency of at least once per day.

13. A method according to claim 8, comprising identifying said subject.

14. A method according to claim 8, wherein said administration is at a frequency of less than once per day.

15. A method comprising:
topically administering to eye/s of a subject in need of treatment for a disorder selected from the group consisting of corneal abrasion, corneal anesthesia and diabetes related neovascularization a composition comprising an effective amount of an active ingredient selected from the group consisting of naltrexone, naloxone or nalmefene or a pharmaceutically acceptable salt thereof and a pharmaceutically acceptable carrier, wherein the composition does not contain moxifloxacin or a salt thereof.

16. A method according to claim 15, wherein the effective amount corresponds to 50-100 microliters at a concentration of at least about 10⁻⁷ Molar.

17. A method according to claim 15, wherein the effective amount corresponds to 50-100 microliters at a concentration of about 10⁻³ Molar or less.

18. A method according to claim 15, wherein said administration is at a frequency of at least once per day.

19. A method according to claim 15, comprising identifying said subject.

20. A method according to claim 15, wherein said administration is at a frequency of less than once per day.
